(19) United States
(12) Patent Application Publication
(10) Pub. No.: US 2022/0168496 A1
(43) Pub. Date: Jun. 2, 2022

(54) EAR IRRIGATING DEVICE

(71) Applicant: Edward David Wagner, Tualatin, OR (US)

(72) Inventor: Edward David Wagner, Tualatin, OR (US)

(21) Appl. No.: 17/105,905

(22) Filed: Nov. 27, 2020

(51) Int. Cl. A61M 3/02

(52) U.S. Cl. CPC A61M 3/0258; A61M 3/0279; A61M 2205/8206; A61M 2210/0662; A61M 2205/42; A61M 2205/3334; A61M 2205/587

(57) ABSTRACT

A device including a water reservoir, in which water reservoir is configured to be operable for storing water. A head portion, in which the head portion contains an ear irrigation tip, a top tubing that is configured to be operable for directing water from the water reservoir to the ear irrigation tip, a wash basin, wherein the wash basin is operable for catching water coming out of the ear irrigation tip, and a splash disk, wherein the splash disk is configured to be operable for redirecting the water and/or debris into the water basin. A main body, in which the main body contains a control interface, wherein the control interface is configured to change a flow rate of the water being pumped out of the ear irrigation tip.

### CROSS-REFERENCE TO RELATED APPLICATIONS

Not applicable.

### RELATED CO-PENDING U.S. PATENT APPLICATIONS

Not applicable.

### INCORPORATION BY REFERENCE OF SEQUENCE LISTING PROVIDED AS A TEXT FILE

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relates to an ear irrigation device. More particularly, certain embodiments of the invention relate to a portable, electric ear irrigation device for the dislodgment and removal of excess ear wax.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Excess ear wax buildup is a common problem that may affect many people throughout the world. Typically, ear wax forms a natural barrier that helps to prevent foreign particles from entering the ear canal, and excess ear wax tends to naturally get dislodged and washed away. However, in many circumstances, excess ear wax may harden and create a blockage in the ear, which may cause discomfort and temporary hearing loss. Attempts to clean the ear or remove excess ear wax from the ear may inadvertently make the problem worse, as ear wax may be pushed deeper into the ear canal and create a blockage. Unremoved earwax may also cause an infection, causing symptoms such as, but not limited to, pain, inflammation, fever, dizziness, etc.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that typical approaches to removing excess ear wax include over the counter ear drops and ear irrigation devices. Often times, ear drops and ear irrigation devices may be used separately or in combination. Excess ear wax may harden, causing a blockage that may be harder to remove. Ear drops may be used to soften the ear wax and gradually help to remove the blockage. This approach may take time and several days to fully remove excess earwax from the ear, and in some circumstances may not be as effective as ear irrigation. Manual ear irrigation may also be used to remove excess earwax from an ear. Typically, a bulb syringe may be used to manually pump water into the ear, irrigating the ear and removing an existing blockage. Improperly irrigating an ear may cause more damage to the ear, and may cause additional hearing loss or infection. For example, without limitation, too much water pressure may cause damage to the ear drum. In many cases, it may be difficult for an individual to clean their own ear, and excess ear wax may be safely removed by a medical professional.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that electric ear irrigation devices may make it easier for an individual to remove excess ear wax from the individual's ears. Typical electric ear irrigation devices may use Piston pumps to pump water into the ear. Piston pumps, however, may be loud, and when used near the ear, may cause additional discomfort for a user. Additionally, typical electric ear irrigation devices may use removable tips that may cause additional damage to the ear if the tip is placed too deeply in the ear, or if the tip is dislodged in the ear.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates an exemplary ear irrigation device, in accordance with an embodiment of the present invention;

FIGS. 2A-2B illustrate an exemplary head portion of an ear irrigation device, wherein FIG. 2A depicts a frontal view of the exemplary head portion and FIG. 2B depicts an exploded view of the exemplary head portion, in accordance with an embodiment of the present invention;

FIG. 3 illustrates a frontal view of an exemplary main body of an ear irrigation device, in accordance with an embodiment of the present invention;

FIG. 4 illustrates a cross section of an exemplary ear irrigation device, in accordance with an embodiment of the present invention;

FIG. 5 illustrates an exemplary water reservoir, in accordance with an embodiment of the present invention; and

FIG. 6 illustrates exemplary internals of an ear irrigation device, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settled law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [”substantially“] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognized in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hollinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” include the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising” And “contain” and variations of them—Such terms are open-ended and mean “including but not limited to”. When employed in the appended claims, this term does not foreclose additional structure or steps. Consider a claim that recites: “A memory controller comprising a system cache . . . . ” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. .sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

All terms of exemplary language (e.g., including, without limitation, “such as”, “like”, “for example”, “for instance”, “similar to”, etc.) are not exclusive of any other, potentially, unrelated, types of examples; thus, implicitly mean “by way of example, and not limitation . . . ”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Moreover, any claim limitation phrased in functional limitation terms covered by 35 USC § 112(6) (post AIA 112(f)) which has a preamble invoking the closed terms “consisting of,” or “consisting essentially of,” should be understood to mean that the corresponding structure(s) disclosed herein define the exact metes and bounds of what the so claimed invention embodiment(s) consists of, or consisting essentially of, to the exclusion of any other elements which do not materially affect the intended purpose of the so claimed embodiment(s).

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

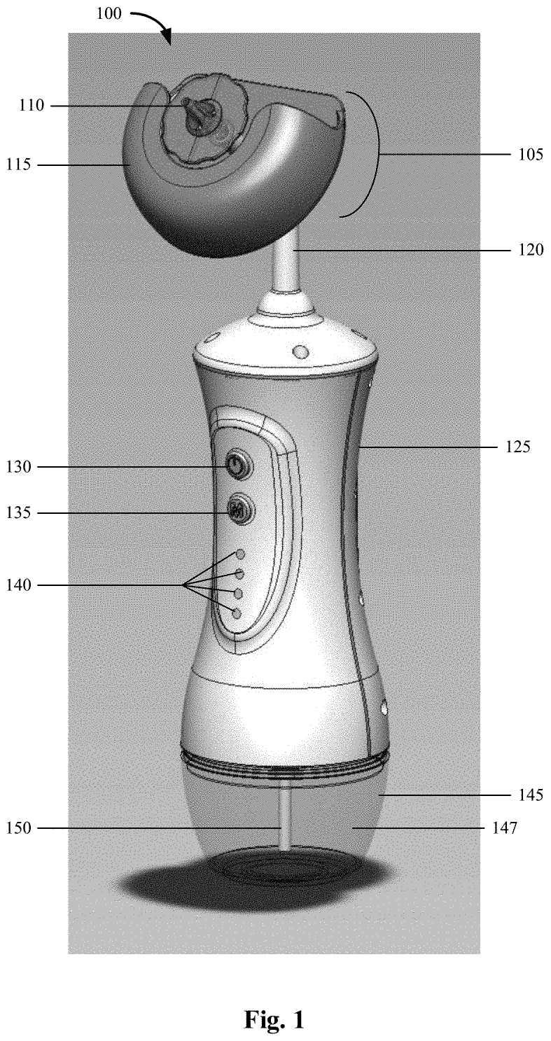

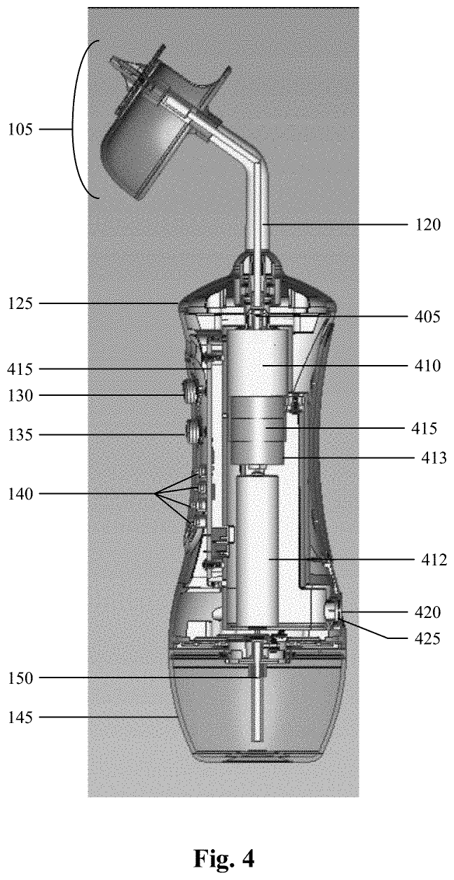

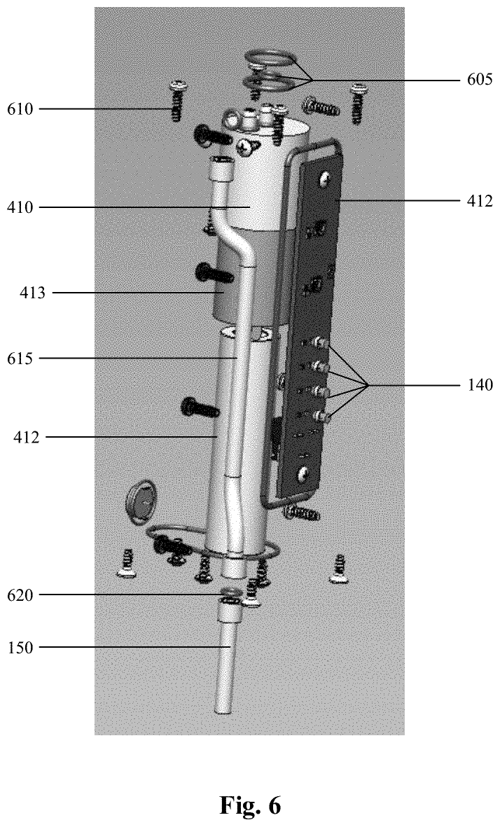

FIG. 1 illustrates an exemplary ear irrigation device, in accordance with an embodiment of the present invention. Ear irrigation device 100 may be composed of three different regions, namely head portion 105, main body 125, and water reservoir 145. Head portion 105 may include ear irrigation tip 110, splash disk, and water or wash basin 115. Ear irrigation tip 110 may comprise of a removable silicon tip. Head portion 105 may be connected to body 125 via top tubing 120. Top tubing 120 may be used as a means for liquid to travel from main body 125 into head portion 105 and out ear irrigation tip 110. Main body 125 may include controls and indicators for controlling ear irrigation device 100, such as, but not limited to, power button 130, mode button 135, and light emitting diodes (LEDs) 140. Main body 125 may also house and protect internal components including a motor/pump (not depicted) which will be described in greater detail with respect to FIG. 4 and FIG. 6 below. Water reservoir 145 may be attached to a bottom portion of body 125, and may include water container 147 to be used to store liquid to be pumped through body 125 and out top portion 105. Water reservoir 145 may further include bottom tubing 150 through which water may be pumped into body 125. Ear irrigation device 100 may be showerproof or waterproof so that ear irrigation device 100 may be used while taking a shower.

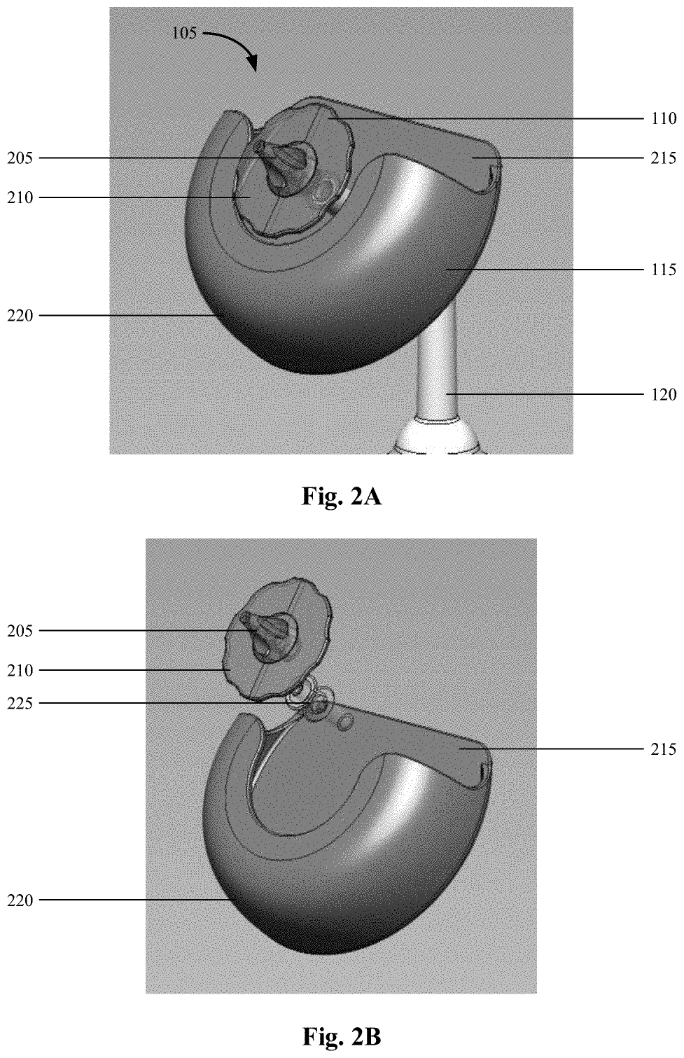

FIGS. 2A-2B illustrate an exemplary head portion of an ear irrigation device, wherein FIG. 2A depicts a frontal view of the exemplary head portion and FIG. 2B depicts an exploded view of the exemplary head portion, in accordance with an embodiment of the present invention. With reference to FIG. 1, FIG. 2A, and FIG. 2B, top portion 105 may be used to efficiently direct liquid into, for example, without limitation, an ear of a user. Top portion 105 may be composed of ear irrigation tip 110 and water basin 115. Ear irrigation tip 105 may include elongation segment 205 and splash disk 210. Elongation segment 205 may be inserted into, for example, without limitation, an ear of a user, and may be used as an efficient means for delivering water into a user's ear. Elongation 205 may be coupled to splash disk 210 such that elongation 205 may be prevented from being inserted too deeply in a user's ear, avoiding potential damage that may occur to a user, as elongation 205 may only be inserted as deeply as splash disk 210. As will be appreciated by one skilled in the art, elongation may be of various lengths depending on the needs of the user. For example, without limitation, elongation segment 205 may be approximately 0.5 to 1 inch in length. Further, the length of elongation 205 may depend on the needs of the user. For example, without limitation, if ear irrigation device 105 is to be used on a younger user, a shorter elongation 205 may be used. Splash disk 210 may be attached to the base of elongation 205, and may be used to redirect water and debris irrigated from the ear of a user away from the user and goes directly into water basin 115. Plastic fitting 225 may be inserted between top tubing 120 and tip 110, creating a water tight seal between top tubing 120 and tip 110 as well as locking tip 110 in place. While tip 110 may be removed and replaced, plastic fitting 225 may be used to ensure that tip 110 may be locked in place and unable to be dislodged during use. Water basin 115 may be composed of back plate 215 and body 115. Water basin 115 may be used with tip 110 to direct water away from the user. Different sizes of water basin 115 may be used depending on the needs of the user. For example, without limitation, water basin 115 may be smaller in size to be used with children or users with smaller ears, and water basin 115 may be larger in size to accommodate for users with larger ears. Water basin 115 may also be swapped out for different sized water basins or other types of attachments to be used with ear irrigation device 100.

Water basin 115 and tip 110 may be composed of, for example, without limitation, single silicon, plastic, synthetic rubber, etc. For example, Dynaflex™ G2755 and G2780 or Versaflex™, which are dishwasher safe and easy to clean. This allows the tip to be reusable. Additionally, water basin 115 and tip 110 may be composed of a transparent material to allow for unobstructed viewing angles in the case water irrigation device 100 may be used on another person. In the preferred embodiment, body 220 of water basin 115 may be semicircular in shape to further allow for better viewing angles when using water irrigation device 100 on another person. As will be appreciated by one skilled in the art, body 220 may be of other shapes, such as, but not limited to, circular, rectangular, hexagonal, irregular, complex, etc.

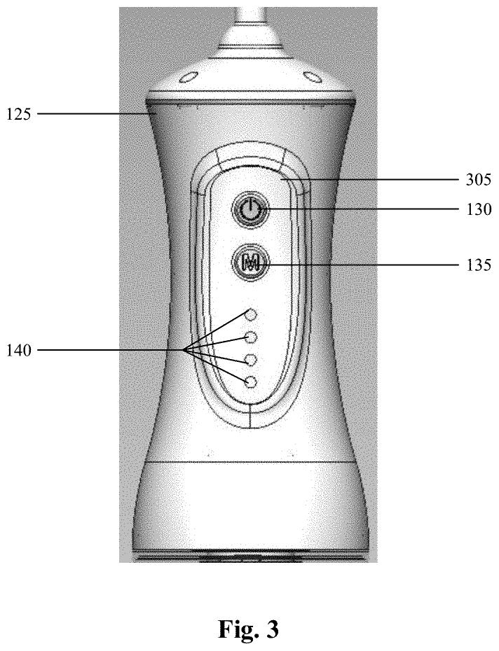

FIG. 3 illustrates a frontal view of an exemplary main body of an ear irrigation device, in accordance with an embodiment of the present invention. With reference to both FIG. 1 and FIG. 3, main body 125 may be used to protect the internal components of ear irrigation device 100, and may be composed of any durable material known in the art, such as, without limitation, plastic, aluminum, and/or synthetic rubber. Additionally, main body 125 may be water proof according to the International Protection (IP) Rating. In the preferred embodiment, water irrigation device 100 may have an IP63 protection rating, allowing a user to use water irrigation device 100 in, for example, the shower, giving the user more freedom as to where they may use water irrigation device 100. Main body 125 may also include control interface 305, wherein controls to operate water irrigation device 100 as well as LEDs that indicate the status of water irrigation device 100 may be located. Control interface 305 may include power button 130, which may be used to power on and off water irrigation device 100. Mode button 135 may be used to change to flow rate of liquid to be pumped out of ear irrigation device 100, allowing a user to adjust the water pressure depending on their needs. Ear irrigation device 100 may have several modes related to flow rate, such as, without limitation, high, medium, low, etc. that may be indicated using LEDs 140. As will be appreciated by one skilled in the art, other means for adjusting the mode of ear irrigation device 100 may be used in combination with or instead of mode button 135. For example, without limitation, the mode of ear irrigation device 100 may be adjusted using a dial, touchscreen, wireless connection to a mobile device, etc. Ear irrigation device 100 may have additional settings for additional features such as, but not limited to, types of water flow (i.e. pulse mode, wherein ear irrigation device 100 may rapidly alternate between turning on and off water flow), selection of different user profiles, etc. Additionally, analog controls may be used to adjust various settings of ear irrigation device 100, allowing a user to have more precise control over the settings of ear irrigation device 100. LEDs 140 may also be used to display other information related to ear irrigation device 100 to the user, such as, without limitation, power status, battery life, water level, warnings, etc. Alternative means for displaying information related to ear irrigation device 100 may also be used, such as, without limitation, digital displays, screens, etc.

FIG. 4 illustrates a cross section of an exemplary ear irrigation device, in accordance with an embodiment of the present invention. With reference to both FIG. 1 and FIG. 4, tip 105 may be connected to main body 125 of ear irrigation device 100 via upper tubing 120. Upper tubing 120 may enter main body 125 and be connected to export end 405 of motor/pump 410. Export end 405 may be used to export water from pump 410 into upper tubing 120. In the preferred embodiment, motor/pump 410 may comprise a water pump or diaphragm pump, providing enough power to pump liquid through upper tubing 120 and producing less noise than a conventional pump. However, as will be appreciated by one skilled in the art, pump 410 may be any type of pump known in the art, such as, but not limited to, piston pumps, plunger pumps, centrifugal pumps, etc. Electrical components, such as, but not limited to, power button 130, mode button 135, LEDs 140, battery casing 413, battery 415, etc. may be coupled to motherboard 412, which provides logistics and control to ear irrigation device 100. In the preferred embodiment, battery 415 may be a lithium ion battery and may comprise one or a plurality of batteries. However, as will be appreciated by one skilled in the art, other types of rechargeable batteries may be used to power ear irrigation device 100, such as, without limitation, nickel cadmium batteries, nickel metal hydride batteries, etc. Additionally, other means of powering ear irrigation device 100 may be used, such as, without limitation, disposable batteries, alternating current (AC) power, etc. To charge battery 415, charging port 420 may be used to connect ear irrigation device 100 to an appropriate charging cable. Charging port 420 may also include magnet connection 425, which may magnetically couple to the charging cable (not shown), providing the user with extra convenience while charging and securing the charging cable in place. Bottom tubing 150 may be connected to water reservoir 145, and may act to provide liquid to ear irrigation device 100.

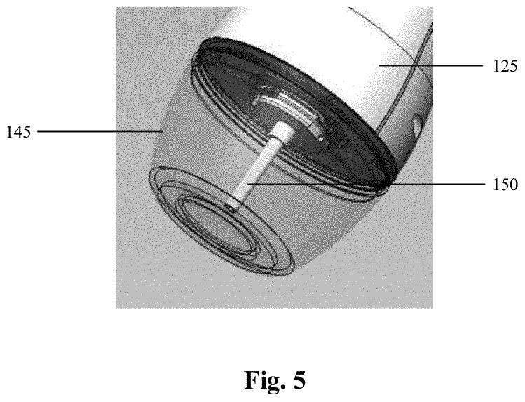

FIG. 5 illustrates an exemplary water reservoir, in accordance with an embodiment of the present invention. With reference to both FIG. 1 and FIG. 5, water reservoir 145 may be attached to the base of main body 125, and may act as a means for storing liquid to be pumped through ear irrigation device 100. Different sized water reservoirs may be used in combination with ear irrigation device 100 depending on the needs of the user. For example, without limitation, water reservoir 145 may be able to hold 2-10 fluid ounces of liquid, depending on the size of water reservoir 145. While water reservoir 145 is depicted as transparent, as will be appreciated by one skilled in the art, water reservoir 145 may also be opaque, semi-opaque, or a combination of transparent, opaque, and semi-opaque. Additionally, the surface of water reservoir 145 may have markings or other such indicators to show the amount of liquid remaining in water reservoir 145. Optionally, an external water reservoir may be used in combination with ear irrigation device 100.

FIG. 6 illustrates exemplary internals of an ear irrigation device, in accordance with an embodiment of the present invention. With reference to FIG. 1, FIG. 4, and FIG. 6, upper O-rings 605 may be attached to a top portion of pump 410 and may act to provide a water tight seal with main body 125 (not shown). A variety of different screws 610 may be used to secure the different parts of ear irrigation device 100 together. Additionally, alternatives to screws 610 may be used, such as, without limitation, rivets, fasteners, clasps, etc. Intermediate tubing 615 may be attached to bottom or lower tubing 150, and may run parallel to pump 410 and battery casing 413 inside of ear irrigation device 100. In-between intermediate tubing 615 and lower tubing 150 may be lower O-ring 620, which may be used to create a water tight seal and ensure that no liquid being pumped through lower tubing 150 into intermediate tubing 615 leaks and causes potential damage to the internal components of ear irrigation device 100.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” or “steps for” claim limitation implies that the broadest initial search on 35 USC § 112(6) (post AIA 112(f)) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC § 112(6) (post AIA 112(f)) when such corresponding structures are not explicitly disclosed in the foregoing patent specification. Therefore, for any invention element(s)/structure(s) corresponding to functional claim limitation(s), in the below claims interpreted under 35 USC § 112(6) (post AIA 112(f)), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC § 112(6) (post AIA 112(f)) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC § 112(6) (post AIA 112(f), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of implementing an ear irrigation device according to the present invention will be apparent to those skilled in the art. Various aspects of the invention have been described above by way of illustration, and the specific embodiments disclosed are not intended to limit the invention to the particular forms disclosed. The particular implementation of ear irrigation device may vary depending upon the particular context or application. By way of example, and not limitation, the ear irrigation device described in the foregoing were principally directed to ear irrigation and ear blockage clearing implementations; however, similar techniques may instead be applied to water based flossing apparatuses, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

Only those claims which employ the words “means for” or “steps for” are to be interpreted under 35 USC 112, sixth paragraph (pre AIA) or 35 USC 112(f) post-AIA. Otherwise, no limitations from the specification are to be read into any claims, unless those limitations are expressly included in the claims.

What is claimed is:

1. A device comprising:
a water reservoir, in which water reservoir comprises;
a water container that is configured to be operable for storing water;
a head portion, in which said head portion comprises;
an ear irrigation tip;
a top tubing that is configured to be operable for directing water from said water reservoir to said ear irrigation tip;
a wash basin, wherein said wash basin is operable for catching water coming out of said ear irrigation tip; and
a splash disk, wherein said splash disk is configured to be operable for redirecting the water and/or debris into said water basin;
a main body, in which said main body comprises;
a control interface, wherein said control interface is configured to change a flow rate of the water being pumped out of said ear irrigation tip.

2. The device of claim 1, further comprising a diaphragm pump that is configured to provide a quiet pumping of the water from said water reservoir to said ear irrigation tip.

3. The device of claim 2, further comprising an on/off button that is configured to turn power on or off to said diaphragm pump.

4. The device of claim 3, in which said device comprises an ear irrigation device and in which said ear irrigation tip comprises a removeable silicon tip.

5. The device of claim 4, in which said water reservoir further comprises a bottom tubing that is configured to carry water to said ear irrigation tip.

6. The device of claim 5, further comprising an elongation segment, where said elongation segment is into engagement with said splash disk, wherein said splash disk is further configured to prevent said ear irrigation tip from being inserted too deeply in a user's ear, avoiding potential damage.

7. The device of claim 6, in which said device further comprises a showerproof device.

8. The device of claim 6, in which said control interface comprises a light emitting diode

9. The device of claim 8, in which said control interface further comprises a mode button that is configured to change the flow rate of water pumped out of said ear irrigation tip.

10. The device of claim 3, further comprising a motherboard, wherein said motherboard is configured to provide logistics and control to said device.

11. The device of claim 10, further comprising a battery casing, wherein said battery casing is configured to hold at least one or more rechargeable or non-rechargeable batteries.

12. The device of claim 11, further comprising a charging port that is configured to charge said one or more rechargeable batteries.

13. The device of claim 12, in which said at least one or more batteries comprise at least one or more of lithium ion batteries, nickel cadmium batteries, and nickel metal hydride batteries.

14. The device of claim 2, further comprising at least one or more upper O-rings that are into engagement with a top portion of said diaphragm pump, wherein said at least one or more upper O-rings are configured to provide a water tight seal of said diaphragm pump with said main body.

15. The device of claim 14, further comprising an intermediate tubing, wherein said intermediate tubing is configured to connect said top tubing and bottom tubing.

16. The device of claim 15, further comprising at least one or more lower O-ring being disposed in-between said intermediate tubing and lower tubing, wherein said lower O-ring is configured to create a water tight seal and ensure that no liquid being pumped through lower tubing into intermediate tubing leaks which may cause potential damage to said components of said device.

17. A device comprising:
means for storing water;
means for irrigating an ear with the water;
means for directing water from said water storing means to said ear irrigating means;
means for catching water coming out of said ear irrigation means;
means for redirecting the water into said water catching means;
means for pumping the water from said water storage means to said ear irrigating means;
means for changing a flow rate of the water being pumped to said ear irrigating means;
means for providing power to said device;
means for charging said power providing means; and
means for providing a water tight seal.

18. A device comprising:
a water reservoir, in which water reservoir comprises:
a water container that is configured to be operable for storing water; and
a bottom tubing that is configured to carry water from said water container to a head portion of said device;
a head portion, in which said head portion comprises;
an ear irrigation tip;
a top tubing that is configured to be operable for directing water from said water reservoir to said ear irrigation tip;
a wash basin, wherein said wash basin is operable for catching water coming out of said ear irrigation tip; and
a splash disk, wherein said splash disk is configured to be operable for redirecting the water and/or debris into said water basin;
a main body, wherein said main body is configured to house and protect internal components of said device.

19. The device of claim 18, in which said internal components comprises:
a diaphragm pump that is configured to provide a quiet pumping of the water from said water reservoir to said ear irrigation tip.
an on/off button that is configured to turn power on or off to said diaphragm pump;
a mode button that is configured to change the flow rate of water pumped out of said ear irrigation tip;
a battery casing, wherein said battery casing is configured to contain at least one or more rechargeable or non-rechargeable batteries;
a charging port that is configured to charge said one or more rechargeable batteries; and
a motherboard, wherein said motherboard is configured to contain said on/off button and mode button.

20. The device of claim 19, in which said device comprises a showerproof ear irrigation device and in which said ear irrigation tip comprises:
a removeable silicon tip; and
an elongation segment, where said elongation segment is into engagement with said splash disk, wherein said splash disk is further configured to prevent said ear irrigation tip from being inserted too deeply in a user's ear, avoiding potential damage.
